(19) United States
(12) Patent Application Publication
(10) Pub. No.: US 2004/0166129 A1
(43) Pub. Date: Aug. 26, 2004

(54) Illumination radiation treatment of skin conditions

(76) Inventors: Robert Marc Clement, Swansea (GB); Michael Noel Kiernan, Swansea (GB)

Correspondence Address: BUDD LARNER ROSENBAUM GREENGERG & SADE, 150 John F. Kennedy Parkway, CN 1000, Short Hills, NJ, 07078-0999, US

(21) Appl. No.: 10/685,840

(22) Filed: Oct. 14, 2003

Related U.S. Application Data
(63) Continuation of application No. 10/399,603.
Application No. 10/399,603 is a 371 of international application No. PCT/GB01/04656, filed on Oct. 18, 2001.

(30) Foreign Application Priority Data
Oct. 18, 2000 (GB) 0025639.6

(51) Int. Cl. A61N 1/30; A61K 7/00

(52) U.S. Cl. 424/401; 604/20

(57) ABSTRACT

A method and apparatus for the cosmetic treatment of a skin condition (particularly Acne Vulgaris). Illuminating radiation (including radiation of a predetermined wavelength) is directed toward a target zone in accordance with a predetermined delivery regime. A plurality of interactions are effected at the target zone including a reaction leading to at least partial disabling or eradication of the cause of the skin condition, and non-ablative heating of tissue stimulating an inflammatory response to a degree sufficient to effect collagen production. The two-fold interaction system effected by the illuminating radiation provides an extremely effective cosmetic effect.

The present invention relates to cosmetic treatment of skin conditions such as, for example, Acne Vulgaris.

Acne Vulgaris is a condition of the sebaceous glands which affects 80% of individuals between the ages of 11 and 30. It is not confined to these age groups however and can affect all ages from neonates to the elderly. Factors which are considered of primary significance to the condition include an increase in the production of sebum, abnormal follicular keratinisation, the presence of Proprionibacterium and subsequent inflammation.

Dependant on the size, content, and depth of the inflamed acne lesion, it is defined as a papule (less than 0.5 cms in diameter), nodule (elevated solid lesion>than 0.5 cms) pustule (a papule what contains purulent material) or a cyst (nodule that contains fluid or semisolid matter).

Hair follicles are minute passages in the skin, which allow hairs to grow and produce sebum secretions from sebaceous glands which are housed within the hair follicle. Due to increased androgen levels or an excessive reaction by the sebaceous glands to androgen production, the sebaceous glands enlarge resulting in increased secretion of sebum which along with the keratinisation process of the epithelial cells, clog the hair follicle. Initially these blockages are microscopic then develop into whiteheads or blackheads (Comedones). Congested follicles are an ideal medium for growth of bacteria. When sebum levels are increased, the skin commensal proprionibacterium ingest the clogged sebum under the skin and chemicals are produced which trigger the immune system to initiate the inflammatory changes and erythematous macules associated with Acne Vulgaris.

Inflammation is the body's response to invasion of pathogens and the redness associated with the acne lesions is the result of increased blood flow whereby the white blood cells invade bacterial cells and damage tissue and produce pus. Other fluids flood to the area and collect at the site of the inflamed tissue.

The approaches which are currently used for tackling Acne Vulgaris include Drug therapies (Systemic antibiotics, cortisone injections, Dianette (women only contraceptive pill, Roaccutane, Retinoids), PUVA (Psoralen and Ultra Violet light, type A), UVB Phototherapy, Dermalux (a system using a combination of red and blue light to treat acne), Peeling agents, laser resurfacing, Dermabrasion and Microdermabrasion.

Known apparatus for treating acne by irradiation are disclosed, for example, in GB2356570, WO00/32272 and U.S. Pat. No. 5,549,660. The present invention provides an improved apparatus and method for the cosmetic treatment of skin conditions (in particular acne).

According to the present invention, there is provided a method for the cosmetic treatment of a skin condition (particularly acne vulgaris), the method comprising directing illuminating radiation, including illuminating radiation of a predetermined wavelength, toward a target zone of skin in accordance with predetermined delivery regime in order to effect at the target zone a plurality of interactions, including:

a) a reaction leading to at least partial disabling or eradication of the cause of the skin condition; and,

b) non-ablative heating of tissue stimulating an inflammatory response to a degree sufficient to effect collagen production.

The two-fold interaction system effected by the illuminating radiation provides an extremely effective cosmetic effect in that the interaction acts to clear up the skin condition and also stimulates the production of collagen to improve skin appearance (minimizing the appearance of scarring caused by the condition). A feature of the technique of the invention is that efficacy is achieved without the need of any other topically applied agent or any invasive or ablative procedure.

It is preferred that the interactions a) and b) defined above occur substantially contemporaneously.

The radiation is typically low intensity (avoiding ablation at or below the skin surface) and typically primarily of wavelength at or about the wavelength of yellow light (585 nm) for reasons explained in detail later. Absorption of light is through the dermal vasculature having no adverse effects on the epidermis.

Desirably, the reaction leading to at least partial removal or disabling of the cause of the skin condition is a photochemical reaction.

Beneficially, the heating interaction is a photothermal effect caused by selective absorption of the predetermined wavelength light, typically by a preselected chromophore.

For Acne Vulgaris the chromophore targeted to combat the proprionibacterium is porphyrin in the connective tissue. This tissue bound photosensitiser when excited from light is of a certain wavelength (approximately the wavelength of yellow light-585 nm), produces a photochemical reaction resulting in the production of singlet oxygen thereby destroying the bacterium.

Proprionibacterium is averse to oxygen (anaerobic) and relies upon chemicals known as porphyrins in skin tissue. Porphyrin is usually innocuous in the absence of light. It is however photosensitive and when exposed to light of the required wavelength the photochemical reaction occurs. This results in a transition from the porphyrin's ground state to a reactive triplet state. At this level, a reaction with molecular oxygen creates singlet oxygen. Through the medium of a suitable light source, to activate the porphyrins to produce singlet oxygen, the bacterium responsible for Acne Vulgaris can be cleared in a cosmetic, pain-free, non-invasive and efficient manner.

Vasodilation and hyperemia are integral parts of the inflammatory response, including response to infection. Therefore any inflammatory/infective focus contains a disproportionate concentration of red blood cells.

Porphyrin molecules are contained in the heam of haemoglobin so that any inflammatory or infective focus contains a concentration of natural porphyrin. Activation of this porphyrin using, for example, yellow (585 nm) light releases substances which destroys adjacent toxins such as bacteria in acne.

Similarly, any acute inflammatory condition of skin such as rosacea will be helped although the exact toxin may be unknown.

Secondly, targeting the chromophore haemoglobin in the dermal vasculature plexus to create thermal injury stimulates the production of fibroblasts which is responsible for collagen production. The stimulated collagen produced is the skin's natural filling material, which will cosmetically improve skin texture and appearance. Exposure to light (of a relevant selected wavelength) results in a selective, non-ablative photothermolysis effect in the target chromophore, that is oxyhaemoglobin. The interaction of the radiation (light) within the dermal vascular plexus induces an inflammatory/growth response. This results in the release of inflammatory mediators from the endothelial cells through the vessel walls and into the dermal interstitium where they stimulate fibroblast activity. Fibroblasts are quiescent unless stimulated by inflammatory mediators. This creates a response by the fibroblasts to initiate tissue repair mechanisms which will in turn produce enhanced new collagen which is the skin's natural filling material and will improve skin texture and appearance.

The energy density of the energy delivered should be accurately controlled and monitored so as not to exceed a predetermined threshold level.

In order to stimulate fibroblast activity, the incident light must be absorbed in the microvasculature to release the necessary mediators which trigger fibroblast activity and hence collagen production.

Certain wavelengths of above, for example, 600 nm (for example, 660 nm-red) are not optimum for collagen stimulation as red light is not preferentially absorbed in haemoglobin/oxyhaemoglobin. An alternative option is to use two wavelengths, one with a high absorption in porphoryin, which has absorption peaks other than those in the yellow region, and at least one wavelength at yellow (570-590 nm).

Where the skin condition is Acne Vulgaris it is therefore preferred that the wavelength of the illuminating radiation comprises a primary wavelength or narrow wavelength band substantially in the range 570-590 nm.

Beneficially the radiation delivered is pulsed, the pulse duration preferably being less than the thermal relaxation time of the target structure. This limits and controls the thermal damage done to the target structure, and controls the correct thermal and chemical response as required.

The photochemical interaction is typically dependent upon the number of incident photons, so the photons may be delivered in pulsed or continuous wave mode. However, for the stimulation of collagen, pulsed operation is preferred to ensure delivery of the required energy regime to cause the triggering of the release of inflammatory mediators.

The light (radiation) source may comprise laser sources (such as laser diodes) or light emitting diodes (LED's) if necessary with appropriate filter(s) to promote propagation of the required selected wavelength (or narrow wavelength band).

Also by using pulsed operation, light emitting devices (particularly LED's) may be driven harder to produce more light output. A typical LED can operate at a drive current of 50 mA in continuous mode, whilst in pulsed operation, for short periods, the same diode can be pulsed at current of around 200 mA. This pulsed operation may be between 1 μs to 100 msec (1 μs to 5 ms preferred). This will allow fewer diodes to be used for a given output power requirement or a larger area to be treated with same amount of diodes.

The target for the light source has to be a material that absorbs a specific wavelength and disregards other wavelengths (chromophore). In accordance with the invention, for Acne Vulgaris, the chromophores may be porphyrin in skin tissue and oxyhaemoglobin in the dermal vasculature.

The preferred wavelength (or wavelengths) for this invention will depend upon the skin condition being treated but typically include a wavelength in the range of 400 nm to 1500 nm with a preferred range of 500-650 nm.

The method according to the present invention is non-invasive and non-ablative and can readily be performed by non-medical personnel. The energy density of the radiation delivered to the skin surface is sufficient to effect the required heating of tissue stimulating an inflammatory response to a degree sufficient to effect collagen production without resulting in unwanted effects on the skin such as ablation and/or other damage. The energy density is therefore preferably substantially in the range of 0.5-5 J/cm² (more preferably substantially in the range of 1.5-3.5 J/cm²) via a pulsed or continuous wave. For pulsed operation the range is lops to looms with a preferred range of 50 μs to 10 ms.

According to a further aspect, the invention provides apparatus for cosmetic treatment of a skin condition (particularly Acne Vulgaris), the apparatus comprising illuminating radiation delivery means for delivering illuminating radiation to a target skin zone or structure.

The apparatus is preferably arranged to output radiation of a discrete wavelength (or narrow primary wavelength band) substantially in or about the range 400 nm-1500 nm, depending upon the skin condition being treated. For treatment of acne vulgaris the preferred range is 500 nm-650 nm, most preferably 570 nm-595 nm.

The apparatus preferably delivers radiation at an energy density at the skin surface substantially in the range 0.5 J/cm²-5 J/cm², (more preferable substantially in the range 1.5-3.5 J/cm²). The apparatus is preferably configured to inhibit output of energies substantially above this range. Desirably the apparatus is configured to permit variable selection of energy densities within the range.

The illuminating radiation may be pulsed or continuous wave. Pulsed energy may be preferred in order to avoid overheating of the target tissue structure (describe above) and produce the appropriate inflammatory response for collagen production. Pulse duration is preferably substantially in the range 10 microseconds-100 ms (preferably substantially in the range 50 microseconds-10 ms).

According to a further, aspect, there is provided a method for the manufacture of an agent for the treatment of a skin condition (particularly Acne Vulgaris), the agent comprising illuminating radiation active to effect at the target zone the following interactions:

a) a reaction leading to at least partial disabling of the cause of the skin condition; and,

b) non-ablative heating of tissue stimulating an inflammatory response to a degree sufficient to effect collagen production.

The invention has been primarily described in relation to the cosmetic treatment of Acne Vulgaris. It will however be appreciated that the two-fold nature of the action described for the invention has potential with respect to other skin conditions, including for example acute inflammatory conditions such as Rosacea, depending upon the selection of the appropriate chromophore/toxin.

The invention will now be further described in specific embodiments, by way of example only and with reference to the accompanying drawings, in which

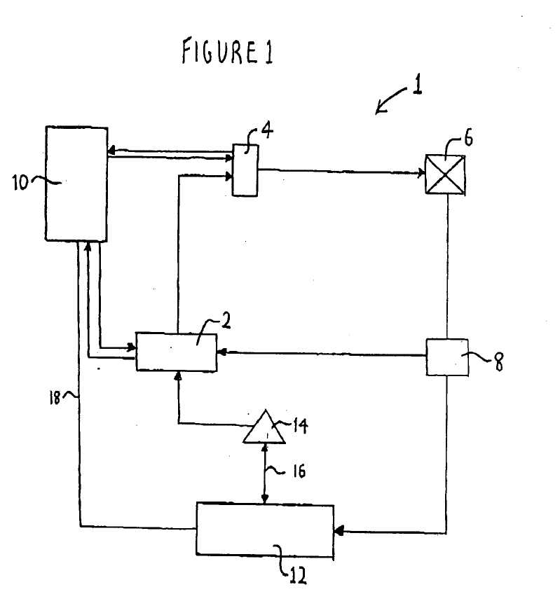

FIG. 1, is a schematic diagram of an embodiment of the apparatus according to the invention;

The apparatus shorn in FIG. 1 comprises a laser radiation delivery system 1. The laser radiation delivery system 1 comprises a flashlamp excited pumped dye laser including a laser head 2, dye reservoir 4, and pump 6.

The system is controlled by a microprocessor controller 12, which operates voltage control of a pulse forming network 14. The pulse forming network 14 includes a capacitor and inductor network. A pulsed beam laser output from laser head 2 is generated by a discharge pulse initiated by the pulse forming network 14. A link 16 provides voltage control and feedback between the microprocessor controller 12 and pulse forming network 14. Also included in system 1 is a flowmeter which, regulates dye flow to the laser cavity in the laser head 2 and a cooling system 10 which cools the laser head 2 and dye reservoir 4. Temperature monitoring feedback is provided between the cooling system 10 and the controller 12 via link 18.

The radiation parameters may be selected to ensure that the total radiation energy density delivered per pulse falls substantially within the range 0.5 J/cm² to 5 J/cm². It is particularly important that the selected upper threshold value (5 J/cm²) is not exceeded significantly as delivery of a higher energy densities of radiation per pulse can result in unwanted effects on the skin (such as ablation and/or other damage). The apparatus is therefore set to ensure an upper threshold permissible level of energy delivered per pulse to be at or below 5 J/cm².

For the dye laser radiation delivery system 1 of FIG. 1, the laser output energy in conjunction with the spot site determines the energy density delivered. The energy density of the radiation delivered to the skin is controlled by adjustment of the flashlamp output energy (which in turn controls the laser output energy). Accurate control is achieved by control of the dye circulation rate, the dye temperature and the flashlamp output energy. Dye circulation rate is important because repeated pulsing of the same volume of dye, without circulation, reduces the output energy of the laser head 2. Increasing or decreasing the dye temperature has an effect on the energy output of the laser head 2. The flashlamp output energy is controlled by varying the voltage with which the capacitors in the pulse forming network 14 are charged; feedback of the capacitor voltage via link 16 is therefore important.

The energy density required will vary within the specified range from person to person, depending upon skin colour.

An alternative embodiment of an apparatus for performance of the present invention utalises an LED or semiconductor laser device to produce output radiation.

High intensity LED devices can provide wavelengths capable of producing the photochemical reaction of porphyrin hereinbefore described (approximately the wavelength of yellow light-585 nm). A radiation delivery system using LED devices may include filters arranged to narrow the band of radiation passing from the LED to the target area of the skin. Where lasers are used, the output may be monochromatic. Alternatively, or in the case where LED's are used, the radiation delivered may be “effectively” monochromatic (for example by means of appropriate filtering) or of a relatively narrow band width (typically within a band width of 15 nm of less).

1. A non-surgical method for the cosmetic treatment of a skin condition comprising directing illuminating radiation toward a target zone of skin in accordance with predetermined delivery regime in order to effect at the target zone a plurality of interactions, including:
(a) a reaction leading to at least partial disabling or eradication of the cause of the skin condition; and
(b) non-ablative heating of tissue stimulating an inflammatory response to a degree sufficient to effect collagen production.

2. A method according to claim 1, for the cosmetic treatment of Acne Vulgaris.

3. A method according to claim 1 or 2, therein the illuminating radiation is of a predetermined wavelength.

4. A method according to any preceding claim, wherein the interactions (a) and (b) occur substantially contemporaneously.

5. A method according to any preceding claim, wherein the reaction leading to at least partial disabling or eradication of the cause of the skin condition is a photochemical reaction.

6. A method according to any preceding claim, wherein the illuminating radiation delivered is pulsed.

7. A method according to claim 6, wherein the pulse duration of the illuminating radiation is less than the thermal relaxation time of the target structure.

8. A method according to claim 6 or 7, wherein the pulse duration of the illuminating radiation is between 10 μs to 100 ms.

9. A method according to claim 8, wherein the pulse duration of the illuminating radiation is between 50 μs to 10 ms.

10. A method according to any preceding claim, wherein the wavelength of the illuminating radiation is in the range of 400 nm to 1500 nm.

11. A method according to claim 10, wherein the illuminating wavelength is in the range 500 nm to 650 nm.

12. A method according to claim 11, wherein the wavelength of the illuminating radiation comprises a primary wavelength or narrow wavelength band substantially in the range 570 nm to 590 nm.

13. A method according to any preceding claim, wherein the energy density of the illuminating radiation is in the range of 0.5 J/cm² to 5 J/cm².

14. A method according to claim 13, wherein the energy density of the illuminating radiation is in the range 1.5 J/cm² to 3.5 cm².

15. Apparatus for cosmetic treatment of a skin condition comprising illuminating radiation delivery means for delivering illuminating radiation to a target skin zone or structure.

16. Apparatus according to claim 15, for cosmetic treatment of Acne Vulgaris.

17. Apparatus according to claim 15 or 16, arranged to output radiation of a discrete wavelength or narrow primary wavelength band substantially in or about the range 400 nm to 1500 nm.

18. Apparatus according to claim 17, arranged to output radiation of a discrete wavelength or narrow primary wavelength band substantially in or about the range 500 nm to 650 nm.

19. Apparatus according to claim 18, arranged to output radiation of a discrete wavelength or narrow primary wavelength band substantially in or about the range 570 nm to 595 nm.

20. Apparatus according to any of claims 15 to 19, arranged to deliver radiation at an energy density at the skin surface substantially in the range 0.5 J/cm² to 5 J/cm².

21. Apparatus according to claim 20, configured to inhibit output of energies substantially above 5 J/cm².

22. Apparatus according to claim 20 or 21, configured to permit variable selection of energy densities within the range 0.5 J/cm² to 5 J/cm².

23. Apparatus according to any preceding claim, arranged to deliver radiation at an energy density at the skin surface substantially in the range 1.5 J/cm² to 3.5 J/cm².

24. Apparatus according to claim 23, configured to inhibit output of energy substantially above 3.5 J/cm².

25. Apparatus according to claim 23 or 24, configured to permit variable selection of energy densities within the range 1.5 J/cm² to 3.5 J/cm².

26. Apparatus according to any of claims 15 to 25, arranged to deliver illuminating radiation in a pulsed regime.

27. Apparatus according to claim 26, wherein the pulse duration of the illuminating radiation is substantially in the range 10 μs to 100 ms.

28. Apparatus according to claim 27, wherein the pulse duration of the illuminating radiation is substantially in the range 50 μs to 10 ms.

29. A method or apparatus for the manufacture of an agent for the treatment of a skin condition, the agent comprising illuminating radiation active to effect at the target zone the following interactions:
(a) a reaction leading to at least partial disabling of the cause of the skin condition; and
(b) non-ablative heating of tissue stimulating an inflammatory response to a degree sufficient to effect collagen production.

30. A method according to claim 29, for the treatment of Acne Vulgaris.
